(19) United States
(12) Patent Application Publication
(10) Pub. No.: US 2009/0036484 A1
(43) Pub. Date: Feb. 5, 2009

(54) Use of Unsaturated Quionoline or Naphtalene Derivatives as Medicaments

(75) Inventor: Hakan Bladh, Lund (SE)

Correspondence Address: FISH & RICHARDSON P.C., P.O BOX 1022, MINNEAPOLIS, MN, 55440-1022, US

(73) Assignee: AstraZeneca AB, Sodertalje (SE)

(21) Appl. No.: 11/718,176

(22) PCT Filed: Oct. 26, 2005
(86) PCT No.: PCT/SE2005/001609

§ 371 Date: Apr. 27, 2007

(30) Foreign Application Priority Data
Oct. 29, 2004 (SE) 0402640-7
Nov. 3, 2004 (SE) 0402678-7

(51) Int. Cl. A61K 31/47; A61P 29/00

(52) U.S. Cl. 514/311

(57) ABSTRACT

A compound of formula (I): (I), or a pharmaceutically acceptable salt thereof; for use as a medicament (for example modulating the glucocorticoid receptor in a warm blooded animal), and pharmaceutical compositions comprising such compounds.

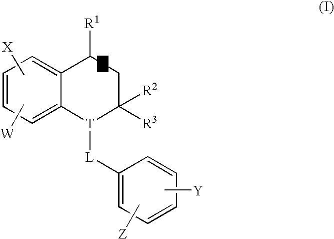

The present invention relates to the use of unsaturated quinoline or naphthalene derivatives as medicaments (for example in the treatment of an inflammatory disease state), to a method of using such derivatives and to pharmaceutical compositions comprising such derivatives.

Hydroquinoline derivatives are disclosed in EP-A1-0347960.

It is known that certain non-steroidal compounds interact with the glucocorticoid receptor (GR) and, as a result of this interaction, produce a suppression of inflammation (see, for example, U.S. Pat. No. 6,323,199). Such compounds can show a clear dissociation between anti-inflammatory and metabolic actions making them superior to earlier reported steroidal and non-steroidal glucocorticoids. The present invention provides further non-steroidal compounds as modulators (for example agonists, antagonists, partial agonists or partial antagonists) of the glucocorticoid receptor capable of having a dissociation between their anti-inflammatory and metabolic actions.

The present invention provides a compound of formula (I):

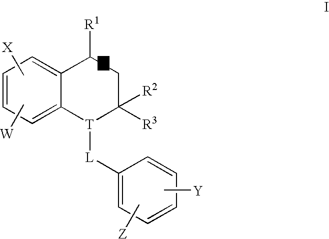

wherein:
T is CH or N;
W, X, Y and Z are, independently, hydrogen, halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR¹⁰DR¹¹; and W and X and/or Y and Z may join to form a fused benzene or pyridine ring;
R¹⁰ and R¹¹ are, independently hydrogen, C₁₋₄ alkyl or C₃₋₇ cycloalkyl;
R¹, R² and R³ are, independently, hydrogen or C₁₋₄ alkyl;
L is CH₂ or C(O);
the bond A is a single or a double bond;
or a pharmaceutically acceptable salt thereof; for use as a medicament.

Compounds of of formula (I) can exist in different isomeric forms (such as enantiomers, diastereomers, geometric isomers or tautomers). The present invention covers the use of all such isomers, and the use of all mixtures of isomers in all proportions.

Suitable salts include acid addition salts such as a hydrochloride, hydrobromide, phosphate, acetate, fumarate, maleate, tartrate, citrate, oxalate, methanesulphonate, p-toluenesulphonate, succinate, glutarate or malonate.

The compounds of formula (I) may exist as solvates (such as hydrates) and the present invention covers the use of all such solvates.

Alkyl groups and moieties are straight or branched chain and are, for example, methyl, ethyl, n-propyl, iso-propyl, n-butyl, sec-butyl or tert-butyl.

Cycloalkyl is for example, cyclopropyl, cyclopentyl or cyclohexyl.

In one particular aspect the present invention provides a compound of formula (I) wherein: W and X are both hydrogen; Y and Z are, independently, hydrogen or halo (such as fluoro or chloro); R¹, R² and R³ are, independently, C₁₋₄ alkyl; L is CH₂ or C(O); the bond A is a single or a double bond; or a pharmaceutically acceptable salt thereof; for use as a medicament.

In another aspect the present invention provides a compound of formula (I) wherein T is N for use as a medicament.

In a further aspect the present invention provides a compound of formula (I) wherein T is CH for use as a medicament.

In a still further aspect the present invention provides a compound of formula (I) wherein W and X are both hydrogen for use as a medicament.

In another aspect the present invention provides a compound of formula (I) wherein Y and Z are, independently, hydrogen or halo (such as fluoro or chloro) for use as a medicament.

In yet another aspect the present invention provides a compound of formula (I) wherein R¹, R² and R³ are, independently, C₁₋₄ alkyl for use as a medicament.

In a further aspect the present invention provides a compound of formula (I) wherein: T is N; W and X are both hydrogen; Y and Z are, independently, hydrogen or halo (such as fluoro or chloro); R¹, R² and R³ are, independently, C₁₋₄ alkyl; L is CH₂ or C(O); the bond A is a single or a double bond; or a pharmaceutically acceptable salt thereof; for use as a medicament.

The compounds of formula (I) can be prepared using or adapting methods disclosed in the art. Starting materials for the preparative methods are either commercially available or can be prepared by literature methods, adapting literature methods.

Because of their ability to bind to the glucocorticoid receptor the compounds of formula (I) are useful as anti-inflammatory agents, and can also display antiallergic, immunosuppressive and anti-proliferative actions. Thus, the compounds of formula (I) can be used as medicaments for treatment or prophylaxis of the following pathologic conditions in mammals (such as humans):
(i) Lung diseases, which coincide with inflammatory, allergic and/or proliferative processes:

chronically obstructive lung diseases of any origin, mainly bronchial asthma

bronchitis of different origins

all forms of restructive lung diseases, mainly allergic alveolitis

all forms of pulmonary edema, mainly toxic pulmonary edema

sarcoidoses and granulomatoses, such as Boeck's disease
(ii) Rheumatic diseases/auto-immune diseases/degenerative joint diseases, which coincide with inflammatory, allergic and/or proliferative processes:

all forms of rheumatic diseases, especially rheumatoid arthritis, acute rheumatic fever, polymyalgia rheumatica, collagenoses

reactive arthritis

inflammatory soft-tissue diseases of other origins

arthritic symptoms in degenerative joint diseases (arthroses)

traumatic arthritides

collagen diseases of other origins, for example systemic lupus erythematodes, sclerodermia, polymyositis, dermatomyositis, polyarteritis nodosa, temporal arteritis

Sjögren's syndrome, Still syndrome, Felty's syndrome
(iii) Allergies, which coincide with inflammatory, allergic and/or proliferative processes:

All forms of allergic reactions, for example Quincke's edema, hay fever, insect bites, allergic reactions to pharmaceutical agents, blood derivatives, contrast media, etc., anaphylactic shock, urticaria, contact dermatitis
(iv) Dermatological diseases, which coincide with inflammatory, allergic and/or proliferative processes:

atopic dermatitis (mainly in children)

psoriasis

erythematous diseases, triggered by different noxae, for example radiation, chemicals, burns, etc.

acid burns

bullous dermatoses

diseases of the lichenoid group

itching (for example of allergic origins)

seborrheal eczema

rosacea

pemphigus vulgaris

erythema exudativum multiforme

erythema nodosum

balanitis

vulvitis

inflammatory hair loss, such as alopecia areata

cutaneous T-cell lymphoma
(v) Nephropathies, which coincide with inflammatory, allergic and/or proliferative processes:

nephrotic syndrome

all nephritides
(vi) Liver diseases, which coincide with inflammatory, allergic and/or proliferative processes:

acute liver cell decomposition

acute hepatitis of different origins, for example virally-, toxically- or pharmaceutical agent-induced

chronically aggressive and/or chronically intermittent hepatitis
(vii) Gastrointestinal diseases, which coincide with inflammatory, allergic and/or proliferative processes:

regional enteritis (Crohn's disease)

ulcerative colitis

gastroenteritis of other origins, for example native sprue
(viii) Proctological diseases, which coincide with inflammatory, allergic and/or proliferative processes:

anal eczema

fissures

haemorrhoids

idiopathic proctitis
(ix) Eve diseases, which coincide with inflammatory, allergic and/or proliferative processes:

allergic keratitis, uvenitis iritis

conjunctivitis

blepharitis

optic neuritis

chorioiditis

sympathetic ophthalmia
(x) Diseases of the ear-nose-throat area, which coincide with inflammatory, allergic and/or proliferative processes:

allergic rhinitis, hay fever

otitis externa, for example caused by contact dermatitis, infection, etc.

otitis media
(xi) Neurological diseases, which coincide with inflammatory, allergic and/or proliferative processes:

cerebral edema, mainly tumor-induced cerebral edema

multiple sclerosis

acute encephalomyelitis

different forms of convulsions, for example infantile nodding spasms
(xii) Blood diseases, which coincide with inflammatory, allergic and/or proliferative processes:

acquired haemolytic anemia

idiopathic thrombocytopenia
(xiii) Tumor diseases, which coincide with inflammatory, allergic and/or proliferative processes:

acute lymphatic leukaemia

malignant lymphoma

lymphogranulomatoses

lymphosarcoma

extensive metastases, mainly in breast and prostate cancers
(xiv) Endocrine diseases, which coincide with inflammatory, allergic and/or proliferative processes:

endocrine orbitopathy

thyrotoxic crisis

de Quervain's thyroiditis

Hashimoto's thyroiditis

hyperthyroidism
(xv) Transplants, which coincide with inflammatory, allergic and/or proliferative processes;
(xvi) Severe shock conditions, which coincide with inflammatory, allergic and/or proliferative processes, for example anaphylactic shock
(xvii) Substitution therapy, which coincides with inflammatory, allergic and/or proliferative processes, with:

innate primary suprarenal insufficiency, for example congenital adrenogenital syndrome

acquired primary suprarenal insufficiency, for example Addison's disease, autoimmune adrenalitis, meta-infective, tumors, metastases, etc.

innate secondary suprarenal insufficiency, for example congenital hypopituitarism

acquired secondary suprarenal insufficiency, for example meta-infective, tumors, etc.
(xviii) Emesis, which coincides with inflammatory, allergic and/or proliferative processes:

for example in combination with a 5-HT₃-antagonist in cytostatic-agent-induced vomiting.

Without prejudice to the foregoing, the compounds of formula (I) can also be used to treat disorders such as: Conies Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, oesophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cinhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis Sweet's disease, type 1 reactive leprosy, capillary hemangiomas, lichen planus, erythema nodosum acne, hirsutism, toxic epidermal necrolysis, erythema multiform, cutaneous T-cell lymphoma, psychoses, cognitive disorders (such as memory disturbances) mood disorders (such as depression and bipolar disorder), anxiety disorders and personality disorders.

As used herein the term “congestive heart failure” (CHF) or ‘congestive heart disease” refers to a disease state of the cardiovascular system whereby the heart is unable to efficiently pump an adequate volume of blood to meet the requirements of the body's tissues and organ systems. Typically, CHF is characterized by left ventricular failure (systolic dysfunction) and fluid accumulation in the lungs, with the underlying cause being attributed to one or more heart or cardiovascular disease states including coronary artery disease, myocardial infarction, hypertension, diabetes, valvular heart disease, and cardiomyopathy. The term “diastolic congestive heart failure” refers to a state of CHF characterized by impairment in the ability of S the heart to properly relax and fill with blood. Conversely, the term “systolic congestive heart failure” refers to a state of CHF characterized by impairment in the ability of the heart to properly contract and eject blood.

As will be appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of physiological disorders contemplates both acute events and chronic conditions. In an acute event, compound is administered at the onset of symptoms and discontinued when the symptoms disappear.

In another aspect the present invention provides the use of a compound or formula (I) or a pharmaceutically acceptable salt thereof, in the manufacture of a medicament for use in the treatment of a glucocorticoid mediated disease state (for example a disease state described above).

In a still further aspect the invention provides the use of a compound of formula (I) or a pharmaceutically acceptable salt thereof, in the manufacture of a medicament for use in the treatment of an inflammatory (such as an arthritic) condition.

In another aspect the invention provides the use of a compound of formula (I) or a pharmaceutically acceptable salt thereof, in the manufacture of a medicament for use in the treatment of an asthmatic condition.

In yet another aspect the invention provides the use of a compound of formula (I) or a pharmaceutically acceptable salt thereof, in the manufacture of a medicament for use in the treatment of a dermatological condition.

In a further aspect the invention provides the use of a compound of formula (I) or a pharmaceutically acceptable salt thereof, in the manufacture of a medicament for use in the treatment of COPD.

In a still further aspect the present invention provides a method of treating a glucocorticoid receptor mediated disease state in a mammal (such as man), which comprises administering to a mammal in need of such treatment an effective amount of a compound of formula (I), or a pharmaceutically acceptable salt thereof.

In another aspect the present invention provides a method of treating an inflammatory (such as an arthritic) condition in a mammal (such as man), which comprises administering to a mammal in need of such treatment an effective amount of a compound of formula (I), or a pharmaceutically acceptable salt thereof.

In yet another aspect the present invention provides a method of treating an asthmatic condition in a mammal (such as man), which comprises administering to a mammal in need of such treatment an effective amount of a compound of formula (I), or a pharmaceutically acceptable salt thereof.

In a further aspect the present invention provides a method of treating a dermatological condition in a mammal (such as man), which comprises administering to a mammal in need of such treatment an effective amount of a compound of formula (I), or a pharmaceutically acceptable salt thereof.

In a still further aspect the present invention provides a method of treating COPD in a mammal (such as man), which comprises administering to a mammal in need of such treatment an effective amount of a compound of formula (I), or a pharmaceutically acceptable salt thereof.

In order to use a compound of formula (I), or a pharmaceutically acceptable salt thereof, for the therapeutic treatment of a mammal, said active ingredient is normally formulated in accordance with standard pharmaceutical practice as a pharmaceutical composition.

Therefore in another aspect the present invention provides a pharmaceutical composition comprising a compound of formula (I), or a pharmaceutically acceptable salt thereof (active ingredient), and a pharmaceutically acceptable adjuvant, diluent or carrier. In a further aspect the present invention provides a process for the preparation of said composition comprising mixing the active ingredient with a pharmaceutically acceptable adjuvant, diluent or carrier. Depending on the mode of administration, the pharmaceutical composition can comprise from 0.05 to 99% w (percent by weight), for example from 0.05 to 80% w, such as from 0.10 to 70% w (for example from 0.10 to 50% w), of active ingredient, all percentages by weight being based on total composition.

A pharmaceutical composition of the present invention may be administered in standard manner for the disease condition that it is desired to treat, for example by topical (such as to the lung and/or airways or to the skin), oral, rectal or parenteral administration. Thus, a the compound of formula (I), or a pharmaceutically acceptable salt thereof, may be formulated into the form of, for example, an aerosol, a powder (for example dry or dispersible), a tablet, a capsule, a syrup, a granule, an aqueous or oily solution or suspension, an (lipid) emulsion, a suppository, an ointment, a cream, drops, or a sterile injectable aqueous or oily solution or suspension.

A suitable pharmaceutical composition of this invention is one suitable for oral administration in unit dosage form, for example a tablet or capsule containing between 0.1 mg and 1 g of active ingredient.

In another aspect a pharmaceutical composition of the invention is one suitable for intravenous, subcutaneous or intramuscular injection.

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. Tablets may be enteric coated by conventional means, for example to provide a coating of cellulose acetate phthalate.

The invention further relates to combination therapies or compositions wherein a compound of formula (I), or a pharmaceutically acceptable salt thereof, or a pharmaceutical composition comprising a compound of formula (I), or a pharmaceutically acceptable salt thereof, is administered concurrently (possibly in the same composition) or sequentially with an agent for the treatment of any one of the above disease states.

In particular, for the treatment of the inflammatory diseases (for example rheumatoid arthritis, COPD, asthma or allergic rhinitis) a compound of the invention can be combined with a TNF-α inhibitor (such as an anti-TNF monoclonal antibody (such as Remicade, CDP-870 and D.sub2.E.sub7.), or a TNF receptor immunoglobulin molecule (such as Enbrel.reg.)), a non-selective COX-1/COX-2 inhibitor (such as piroxicam or diclofenac; a propionic acid such as naproxen, flubiprofen, fenoprofen, ketoprofen or ibuprofen; a fenamate such as mefenamic acid, indomethacin, sulindac or apazone; a pyrazolone such as phenylbutazone; or a salicylate such as aspirin), a COX-2 inhibitor (such as meloxicam, celecoxib, rofecoxib, valdecoxib or etoricoxib) low dose methotrexate, lefunomide; ciclesonide; hydroxychloroquine, d-penicillamine or auranofin, or parenteral or oral gold.

The present invention still further relates to the combination of a compound of the invention together with:

a leukotriene biosynthesis inhibitor, a 5-lipoxygenase (5-LO) inhibitor or a 5-lipoxygenase activating protein (FLAP) antagonist, such as zileuton, ABT-761, fenleuton, tepoxalin, Abbott-79175, Abbott-85761, an N-(5-substituted)-thiophene-2-alkylsulfonamide, a 2,6-di-tert-butylphenol hydrazones, a methoxytetrahydropyran such as Zeneca ZD-2138, SB-210661, a pyridinyl-substituted 2-cyanonaphthalene compound such as L-739,010; a 2-cyanoquinoline compound such as L-746,530; an indole or quinoline cornpound such as MK-591, MK-886 or BAY x 1005;

a receptor antagonist for a leukotriene LTB.sub4., LTC.sub4., LTD.sub4. or LTE.sub4. selected from the group consisting of a phenothiazin-3-one such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A) or BAY x 7195;

a PDE4 inhibitor including an inhibitor of the isoform PDE4D;

an antihistaminic H.sub1. receptor antagonist such as cetirizine, loratadine, desloratadine, fexofenadine, astemizole, azelastine or chlorpheniramine;

a gastroprotective H.sub2. receptor antagonist;

an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride or ethylnorepinephrine hydrochloride;

an anticholinergic agent such as ipratropium bromide, tiotropium bromide, oxitropium bromide, pirenzepine or telenzepine;

a β.sub1.- to β.sub4.-adrenoceptor agonist (such as β2 adrenoceptor agonist) such as metaproterenol, isoproterenol, isoprenaline, albuterol, salbutamol, formoterol, salmeterol, terbutaline, orciprenaline, bitolterol mesylate or pirbuterol, or a methylxanthanine including theophylline and aminophylline; sodium cromoglycate; or a muscarinic receptor (M1, M2, and M3) antagonist;

an insulin-like growth factor type I (IGF-1) mimetic;

an inhaled glucocorticoid with reduced systemic side effects, such as prednisone, prednisolone, flunisolide, triamcinolone acetonide, beclomethasone dipropionate, budesonide, fluticasone propionate or mometasone furoate;

an inhibitor of a matrix metalloprotease (MMP), such as a stromelysin, a collagenase, or a gelatinase or aggrecanase; such as collagenase-1 (MMP-1), collagenase-2 MMP-8), collagenase-3 (MMP-13), stromelysin-1 (MMP-3), stromelysin-2 (MMP-10), and stromelysin-3 (MMP-11) or MMP-12;

a modulator of chemokine receptor function such as CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family;

an osteoporosis agent such as roloxifene, droloxifene, lasofoxifene or fosomax;

an immunosuppressant agent such as FK-506, rapamycin, cyclosporine, azathioprine or methotrexate;

a compound useful in the treatment of AIDS and/or HIV infection for example: an agent which prevents or inhibits the viral protein gp120 from engaging host cell CD4 {such as soluble CD4 (recombinant); an anti-CD4 antibody (or modified/recombinant antibody) for example PRO542; an anti-group 120 antibody (or modified / recombinant antibody); or another agent which interferes with the binding of group 120 to CD4 for example BMS806}; an agent which prevents binding to a chemokine receptor, other than CCR5, used by the HIV virus {such as a CXCR4 agonist or antagonist or an anti-CXCR4 antibody}; a compound which interferes in the fusion between the HIV viral envelope and a cell membrane {such as an anti-group 41 antibody; enfuvirtide (T-20) or T-1249}; an inhibitor of DC-SIGN (also known as CD209) {such as an anti-DC-SIGN antibody or an inhibitor of DC-SIGN binding}; a nucleoside/nucleotide analogue reverse transciptase inhibitor {for example zidovudine (AZT), nevirapine, didanosine (ddI), zalcitabine (ddC), stavudine (d4T), lamivudine (3TC), abacavir, adefovir or tenofovir (for example as free base or as disoproxil fumarate)}; a non-nucleoside reverse transciptase inhibitor {for example nevirapine, delavirdine or efavirenz}; a protease inhibitor {for example ritonavir, indinavir, saquinavir (for example as free base or as mesylate salt), nelfinavir (for example as free base or as mesylate salt), amprenavir, lopinavir or atazanavir (for example as free base or as sulphate salt)}; a ribonucleotide reductase inhinbitor {for example hydroxyurea}; or an antiretroviral {for example emtricitabine}; or, an existing therapeutic agent for the treatment of osteoarthritis, for example a non-steroidal anti-inflammatory agent (hereinafter NSAID's) such as piroxicam or diclofenac, a propionic acid such as naproxen, flubiprofen, fenoprofen, ketoprofen or ibuprofen, a fenamate such as mefenamic acid, indomethacin, sulindac or apazone, a pyrazolone such as phenylbutazone, a salicylate such as aspirin, a COX-2 inhibitor such as celecoxib, valdecoxib, rofecoxib or etoricoxib, an analgesic or intra-articular therapy such as a corticosteroid or a hyaluronic acid such as hyalgan or synvisc, or a P2X7 receptor antagonist.

The present invention still further relates to the combination of a compound of the invention together with: (i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitor including a VLA-4 antagonist; (vi) a cathepsin; (vii) a MAP kinase inhibitor; (viii) a glucose-6 phosphate dehydrogenase inhibitor; (ix) a kinin-B.sub1.- and B.sub2.-receptor antagonist; (x) an anti-gout agent, e.g., colchicine; (xi) a xanthine oxidase inhibitor, e.g., allopurinol; (xii) an uricosuric agent, e.g., probenecid, sulfmpyrazone or benzbromarone; (xiii) a growth hormone secretagogue; (xiv) a transforming growth factor (TGFβ); (xv) a platelet-derived growth factor (PDGF); (xvi) a fibroblast growth factor, e.g., basic fibroblast growth factor (bFGF); (xvii) a granulocyte macrophage colony stimulating factor (GM-CSF); (xviii) a capsaicin cream; (xix) a Tachykinin NK.sub1. and NK.sub3. receptor antagonist selected from the group consisting of NKP-608C; SB-233412 (talnetant); and D-4418; (xx) an elastase inhibitors selected from the group consisting of UT-77 and ZD-0892; (xxi) a TNFα converting enzyme inhibitor (TACE); (xxii) an induced nitric oxide synthase inhibitor (iNOS); or (xxiii) a chemoattractant receptor-homologous molecule expressed on TH2 cells (a CRTH2 antagonist).

The following compounds illustrate compounds of formula (I) and are accompanied by their Chemical Abstracts Registry Numbers.

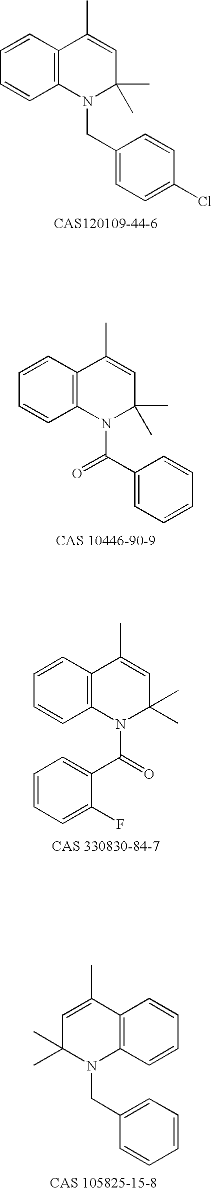

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10× (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10× (Panvera, Part number P2814, stored at −70° C. initially but once thawed stored at room temperature). Avoid repeated freeze/thaws for all reagents. The GR Screening buffer 10× comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (8 μL; assay buffer 10×, Stabilizing Peptide, DTT and ice cold MQ water) was added to the background wells. GS Red solution (7 μL; assay buffer 10×, Stabilizing Peptide, DTT, GS Red and ice cold water) was added to all wells except background wells. GR solution (7 μL; assay buffer 10×, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nM and a dichroic mirror at 561 nm). The IC50 values were calculated using XLfit model 205.

| Compound | IC₅₀ (nM) |
|---|---|
| CAS120109-44-6 | 240 |

1. A pharmaceutical composition comprising a compound of formula (I). 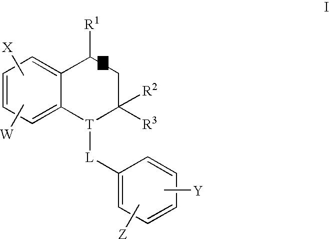
wherein:
T is CH or N;
W, X, Y and Z are, independently, hydrogen, halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR¹⁰R¹¹; and W and X and/or Y and Z may join to form a fused benzene or pyridine ring;
R¹⁰ and R¹¹ are, independently hydrogen, C₁₋₄ alkyl or C₃₋₇ cycloalkyl;
R¹, R² and R³ are, independently, hydrogen or C₁₋₄ alkyl;
L is CH₂ or C(O);
the bond A is a single or a double bond;
or a pharmaceutically acceptable salt thereof;
and a pharmaceutically acceptable adjuvant, diluent or carrier.

2. The pharmaceutical composition of claim 1 wherein T is N.

3. The pharmaceutical composition of claim 1 wherein T is CH.

4. The pharmaceutical composition of claim 1 wherein W and X are both hydrogen.

5. The pharmaceutical composition of claim 1 wherein Y and Z are, independently, hydrogen or halo.

6. The pharmaceutical composition of claim 1 wherein R¹, R² and R³ are, independently, C₁₋₄ alkyl.

7. The pharmaceutical composition of claim 1 wherein: T is N; W and X are both hydrogen; Y and Z are, independently, hydrogen or halo; R¹, R² and R³ are, independently, C₁₋₄ alkyl; L is CH₂ or C(O); the bond A is a single or a double bond.

8. (canceled)

9. A method of treating a glucocorticoid receptor mediated disease state in a mammal, which comprises administering to a mammal in need of such treatment an effective amount of a compound of formula (I), or a pharmaceutically acceptable salt thereof 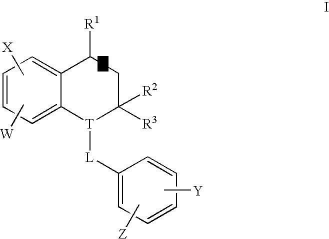
wherein:
T is CH or N;
W, X, Y and Z are, independently, hydrogen, halo, C₁₋₄ alkyl, C₁₋₄ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR¹⁰R¹¹; and W and X and/or Y and Z may join to form a fused benzene or pyridine ring;
R¹⁰ and R¹¹ are, independently hydrogen, C₁₋₄ alkyl or C₃₋₇ cycloalkyl;
R¹, R² and R³ are, independently, hydrogen or C₁₋₄ alkyl;
L is CH₂ or C(O);
the bond A is a single or a double bond.

10. (canceled)
